(19) United States
(12) Patent Application Publication
(10) Pub. No.: US 2004/0133000 A1
(43) Pub. Date: Jul. 8, 2004

(54) Process for the preparation of oxabispidines

(76) Inventors: Lal Cheema, Loughborough (GB); David Cladingboel, Loughborough (GB)

Correspondence Address: ROPES & GRAY LLP, ONE INTERNATIONAL PLACE, BOSTON, MA, 02110-2624, US

(21) Appl. No.: 10/474,585

(22) PCT Filed: Apr. 12, 2002
(86) PCT No.: PCT/SE02/00728

(30) Foreign Application Priority Data
Apr. 12, 2001 (SE) 0101323-4

(51) Int. Cl. C07D 491/04

(52) U.S. Cl. 544/74

(57) ABSTRACT

There is provided a process for the preparation of a benzenesulfonic acid salt of a compound of formula (1) which process comprises reaction of a compound of formula (II) with a compound of formula (III) wherein R₁, R₂, A and B have meanings given in the description.

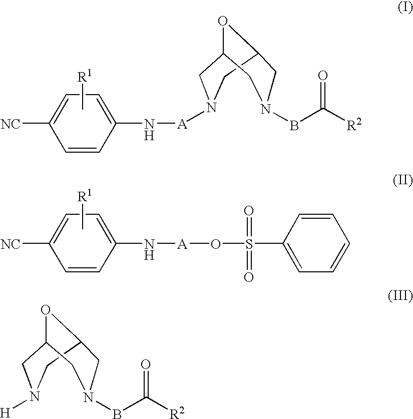

### FIELD OF THE INVENTION

This invention relates to a novel process for the preparation of N-ketoalkyl-N′-anilinoalkyl oxabispidine benzenesulfonic acid salts.

### PRIOR ART

The number of documented compounds including the 9-oxa-3,7-diazabicyclo-[3.3.1]nonane (oxabispidine) structure is very few. As a result, there are very few known processes that are specifically adapted for the preparation of oxabispidine compounds.

Certain oxabispidine compounds are disclosed in Chem. Ber. 96(11), 2827 (1963) as intermediates in the synthesis of 1,3-diaza-6-oxa-adamantanes.

Hemiacetals (and related compounds) having the oxabispidine ring structure are disclosed in J. Org. Chem. 31, 277 (1966), ibid. 61(25), 8897 (1996), ibid. 63(5), 1566 (1998) and ibid. 64(3), 960 (1999) as unexpected products from the oxidation of 1,5-diazacyclooctane-1,3-diols or the reduction of 1,5-diazacyclooctane-1,3-diones.

1,3-Dimethyl-3,7-ditosyl-9-oxa-3,7-diazabicyclo[3.3.1]nonane is disclosed in J. Org. Chem. 32, 2425 (1967) as a product from the attempted acetylation of trans-1,3-dimethyl-1,5-ditosyl-1,5-diazacyclooctane-1,3-diol.

None of the above-mentioned documents disclose or suggest the synthesis of oxabispidines bearing a ketoalkyl substituent on one N-atom and an anilinoalkyl substituent on the other.

International patent application WO 01/28992 describes the synthesis of a wide range of oxabispidine compounds, which compounds are indicated as being useful in the treatment of cardiac arrhythmias. Amongst the compounds disclosed is 4-({3-[7-(3,3-dimethyl-2-oxobutyl)-9-oxa-3,7-diazabicyclo[3.3.1]non-3-yl]propyl}amino)benzonitrile, benzenesulfonic acid salt (isolated as the monohydrate). However, in the route disclosed in WO 01/28992 for the preparation of that salt, the product is formed via the coupling of 3-(4-cyanoanilino)propyl 4-methylbenzenesulfonate to the oxabispidine nucleus, followed by anion exchange of 4-methylbenzenesulfonate for benzenesulfonate.

We have now found, surprisingly, that benzenesulfonic acid salts of N-ketoalkyl-N′-anilinoalkyl oxabispidines may be conveniently prepared directly by reaction between N-ketoalkyl oxabispidines and anilinoalkylyl benzenesulfonates.

### DESCRIPTION OF THE INVENTION

According to a first aspect of the invention there is provided a process for the preparation of a benzenesulfonic acid salt of a compound of formula I,

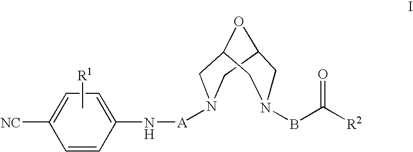

wherein R¹ represents H or cyano;

A represents (CH₂)₂₋₆;

B represents (CH₂)₁₋₄; and

R² represents C₁₋₆ alkyl, phenyl (which latter group is optionally substituted by one or two substituents selected from halo and methoxy) or benzodioxanyl;

which process comprises reaction of a compound of formula II,

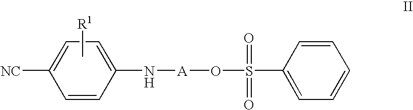

wherein R¹ and A are as defined above, with a compound of formula III,

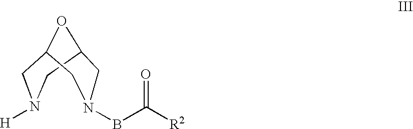

wherein B and R² are as defined above,

and which process is referred to hereinafter as “the process of the invention”.

Unless otherwise specified, alkyl groups as defined herein may be straight-chain or, when there is a sufficient number (i.e. a minimum of three) of carbon atoms, be branched-chain and/or cyclic. Further, when there is a sufficient number (i.e. a minimum of four) of carbon atoms, such alkyl groups may also be part cyclic/acyclic. Such alkyl groups may also be saturated or, when there is a sufficient number (i.e. a minimum of two) of carbon atoms, be unsaturated. Unless otherwise specified, alkyl groups may also be substituted by one or more halo, and especially fluoro, atoms. The term “halo”, when used herein, includes fluoro, chloro, bromo and iodo. Preferred values of R¹ include cyano (for example located at the ortho-position relative to the group —N(H)-A-) and, particularly, H.

Preferred values of A include (CH₂)₂₋₄, and, particularly n-propylene.

Preferred values of B include (CH₂)₁₋₃, and, particularly, CH₂.

Preferred values of R² include benzodioxan-6-yl, 4-fluorophenyl, 4-bromo-phenyl, 4-methoxyphenyl, 3,4-dimethoxyphenyl and, particularly C₁₋₄ alkyl (such as methyl and, particularly, tert-butyl).

The process of the invention is preferably carried out in the presence of a suitable solvent system. This solvent system should not give rise to stereochemical changes in the reactants or product once formed.

Suitable solvents include polar organic solvents (e.g. DMF, N-methyl-pyrrolidinone or acetonitrile) or, preferably, hydroxylic solvents such as lower alkyl alcohols (e.g. C₁₋₄ alcohols such as ethanol) and/or water. It is preferred that the process is carried out in the presence of ethanol as solvent.

It is also preferred that once reaction is complete, the compound of formula I is subsequently precipitated from solution. It is further preferred that this precipitation is facilitated by the addition of water to the reaction mixture.

The process of the invention is preferably carried out at, or above, ambient temperature, such as at between room temperature and reflux temperature of the solvent that is employed (e.g. between 10 and 100° C., preferably between 15 and 90° C., and particularly between 20 and 80° C.). For example, when the solvent that is employed is ethanol, the reaction may be carried out at around reflux temperature (such as between 70 and 80° C., and, particularly, 74° C.).

In the process of the invention, the stoichiometric ratio of the compound of formula II to the compound of formula III is preferably within the range of 3:2 to 2:3, particularly within the range 5:4 to 4:5 (such as within the range 11:10 to 10:11), and, especially, 1:1.

The benzenesulfonate salt of the compound of formula I, when obtained by the process of the invention, may subsequently be purified by conventional techniques, such as recrystallisation. Suitable solvents for the recrystallisation procedure include lower alkyl alcohols (e.g. C₁₋₄ alcohols such as ethanol), water and mixtures thereof The preferred recrystallisation solvent is ethanol/water. As will be appreciated by those skilled in the art, the use of higher solvent volumes during recrystallisation, although providing a lower recovery of recrystallised product, may yield a product of higher purity than that obtained when lower solvent volumes are used. Thus, the volume of solvent used in the recrystallisation may be selected in accordance with the degree of purity that is desired for the recrystallised product.

Compounds of formula II may be prepared using conventional techniques. For example, compounds of formula II may be prepared by reaction of a corresponding compound of formula IV,

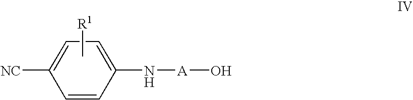

wherein R¹ and A are as hereinbefore defined, with benzenesulfonyl chloride, for example at between −25° C. and room temperature in the presence of a suitable base (e.g. a tertiary amine such as triethylamine), an appropriate solvent (e.g. acetonitrile, toluene or, preferably, CH₂Cl₂) and optionally in the presence of a suitable catalyst (e.g. 4-(dimethylamino)-pyridine or, preferably, a tertiary amine acid addition salt such as trimethylamine hydrochloride (see Tetrahedron 55, 2183 (1999)).

Compounds of formula III may be prepared by reaction of 9-oxa-3,7-diazabicyclo[3.3.1]nonane (formula V),

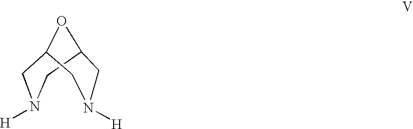

or a N-protected derivative thereof, with a compound of formula VI,

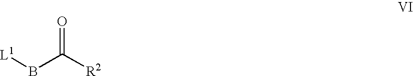

wherein L¹ represents a suitable leaving group (e.g. halo, such as chloro) and B and R² are as hereinbefore defined, for example at between room temperature and 70° C. in the presence of a suitable base (e.g. an alkali or alkaline earth metal hydroxide, carbonate or hydrogencarbonate, such as NaHCO₃) and an appropriate solvent (e.g. a lower alkyl (e.g. C₁₋₆) alcohol (such as ethanol) or, particularly, water).

Compounds of formula IV are known in the art or may be prepared using known techniques. For example, compounds of formula IV may be prepared by reaction of a corresponding compound of formula VII,

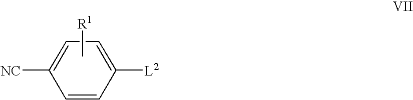

wherein L² represents a suitable leaving group (e.g. fluoro) and R¹ is as hereinbefore defined, with a compound of formula VIII,

H₂N-A-OH VIII

wherein A is as hereinbefore defined, for example at between room temperature and 80° C. in the presence of an excess of the compound of formula VIII (which compound may also act as a solvent for the compound of formula VII (in this reaction).

9-Oxa-3,7-diazabicyclo[3.3.1]nonane (the compound of formula V) and N-protected derivatives thereof may be prepared by dehydrative cyclisation of 3,7-dihydroxy-1,5-diazacyclooctane (the compound of formula IX),

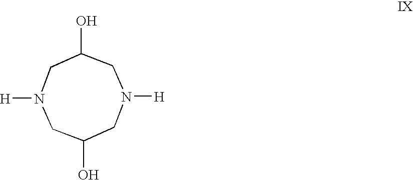

or a N-protected derivative thereof, wherein R¹ is as hereinbefore defined. This cyclisation may be carried out, for example in the presence of a suitable dehydrating agent (such as: a strong acid (e.g. sulfuric acid (e.g. concentrated sulfuric acid) or, particularly, methanesulfonic acid (especially anhydrous methanesulfonic acid) and the like); an acid anhydride such as acetic anhydride or trifluoromethane-sulfonic anhydride; P₂I₅ in methanesulfonic acid; a phosphorous-based halogenating agent such as P(O)Cl₃, PCl₃ or PCl₅; or thionyl chloride). The cyclisation may also be carried out in the presence of a suitable organic solvent system, which solvent system should not significantly react chemically with, or significantly give rise to stereochemical changes in, the reactant or product once formed, or significantly give rise to other side reactions. Preferred solvent systems include aromatic solvents (e.g. an aromatic hydrocarbon, such as toluene or xylene, or a chlorinated aromatic hydrocarbon, such as chlorobenzene or dichlorobenzene), or dichloroethane, optionally in the presence of further solvents such as ethanol and/or ethyl acetate. When the dehydrating agent is methanesulfonic acid, preferred solvent systems include toluene. When the dehydrating agent is sulfuric acid, preferred solvent systems include chlorobenzene or no solvent. The cyclisation may be carried out at elevated temperature (e.g. up to the reflux temperature of the relevant solvent system, or higher if a pressurised system is employed). Clearly, appropriate reaction times and reaction temperatures depend upon the solvent system that is employed, but these may be determined routinely by the skilled person.

9-Oxa-3,7-diazabicyclo[3.3.1]nonane (the compound of formula V) and N-protected derivatives thereof may alternatively be prepared according to, or by analogy with, known techniques, for example by reaction of a compound of formula X,

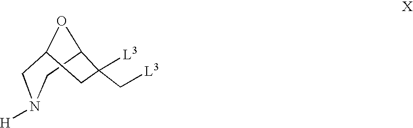

or an N-protected derivative thereof, wherein L³ represents a suitable leaving group (e.g. halo, such as iodo), with ammonia or a protected derivative thereof (e.g. benzylamine), for example under conditions such as those described in Chem. Ber. 96(1.1), 2827 (1963).

3,7-Dihydroxy-1,5-diazacyclooctane (the compound of formula IX) and N-protected derivatives thereof may be prepared by reaction of bis(2-oxiranylmethyl)amine (the compound of formula XI),

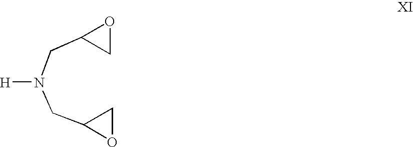

or a N-protected derivative thereof, with ammonia or a protected derivative thereof (e.g. benzylamine), for example at between room temperature and the reflux temperature of any solvent that is employed (preferably at or around reflux temperature). Suitable solvent systems that may be employed include organic solvent systems, which systems should not significantly react chemically with, or significantly give rise to stereochemical changes in, the reactants or product once formed, or significantly give rise to other side reactions. Preferred solvent systems include hydroxylic compounds such as ethanol, methanol, propan-2-ol, or mixtures thereof (such as industrial methylated spirit (IMS)), optionally in the presence of an appropriate co-solvent (e.g. an ester, such as ethyl acetate, an aromatic solvent, such as toluene or chlorobenzene, or water). Preferred solvents for this reaction include primary alcohols such as methanol, propanol and, especially, ethanol, and preferred co-solvents include toluene and chlorobenzene.

Compounds of formula X may be prepared by known techniques, for example according to or by analogy with the procedures described in Chem. Ber. 96(11), 2827 (1963) and international patent application WO 01/28992.

Bis(2-oxiranylmethyl)amine (the compound of formula XI) and N-protected derivatives thereof may be prepared by reaction of two or more equivalents of a compound of formula XII,

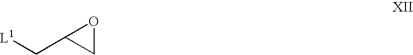

wherein L¹ is as hereinbefore defined, with ammonia, or a N-protected derivative thereof, for example at between room and reflux temperature in the presence of a suitable base (e.g. an alkali metal carbonate such as cesium carbonate, sodium hydroxide, sodium hydride or lithium diisopropylamide), an appropriate solvent (e.g. acetonitrile, N,N-dimethylformamide, THF, toluene, water or mixtures thereof), and optionally in the presence of a phase transfer catalyst (e.g. tricaprylylmethylammonium chloride). Preferred bases include sodium hydroxide and preferred solvents include water.

Compounds of formulae VI, VII, VIII and XII, and derivatives thereof, are either commercially available, are known in the literature or may be obtained by analogy with the processes described herein, or by conventional synthetic procedures, in accordance with standard techniques, from readily available starting materials using appropriate reagents and reaction conditions.

It will be appreciated by those skilled in the art that, in the processes described above, the functional groups of intermediate compounds may be, or may need to be, protected by protecting groups.

Functional groups which it is desirable to protect include hydroxy and amino. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include benzyl, sulfonyl (e.g. benzenesulfonyl or nitrobenzenesulfonyl), tert-butyloxycarbonyl, 9-fluorenylmethoxy-carbonyl or benzyloxycarbonyl.

In particular, it may be desirable to protect:

(i) one amino group of 9-oxa-3,7-diazabicyclo[3.3.1]nonane (the compound of formula V) with an appropriate protecting group (such as benzyl), which should be removed after reaction with the compound of formula VI to form a compound of formula III;

(ii) one or both of the amino groups of 3,7-dihydroxy-1,5-diazacyclooctane (the compound of formula IX) with appropriate protecting groups (such as benzyl (one one side) and benzenesulfonyl or nitrobenzenesulfonyl, such as a N-4-nitrobenzenesulfonyl (on the other)). If two protecting groups are employed, then at least one of these should be removed after the protected 9-oxa-3,7-diazabicyclo-[3.3.1]nonane (the compound of formula V) is formed (e.g. if a benzyl group, and a benzenesulfonyl/nitrobenzenesulfonyl group, are employed to protect the two amino groups, the benzenesulfonyl/nitrobenzenesulfonyl group may be removed after the N-protected compound of formula V is formed, prior to reaction of that compound with a compound of formula VI);

(iii) the amino group of a compound of formula X with an appropriate protecting group (such as benzenesulfonyl), which should be removed after the compound of formula V is formed; and/or

(iv) the amino group of bis(2-oxiranylmethyl)amine (the compound of formula XI) with an appropriate protecting group (such as benzenesulfonyl or nitrobenzenesulfonyl (e.g. N4-nitrobenzenesulfonyl)), which should be removed after the N-protected compound of formula V is formed.

The protection and deprotection of functional groups may take place before or after any of the reaction steps described hereinbefore.

Protecting groups may be removed in accordance with techniques which are well known to those skilled in the art and as described hereinafter.

The use of protecting groups is fully described in “Protective Groups in Organic Chemistry”, edited by J. W. F. McOmie, Plenum Press (1973), and “Protective Groups in Organic Synthesis”, 3rd edition, T. W. Greene & P. G. M. Wutz, Wiley-Interscience (1999).

The process of the invention possesses the surprising advantage that compounds of formula I may be obtained in a simple, ‘one-pot’ procedure from compounds of formula III without the need for subsequent anion exchange (which may involve neutralisation and solvent exchange). This provides the further advantage that the introduction of impurities from the reagents that would need to be employed during an anion exchange process is avoided. Thus, the need to utilise very pure materials in such a process is also avoided.

Further, the process of the invention may have the advantage that compounds of formula I may be prepared in higher yields, in less time, more conveniently, and at a lower cost, than when prepared according to any process that may be described in the prior art.

The invention is illustrated, but in no way limited, by the following examples.

### EXAMPLES

General Experimental Procedures

Mass spectra were recorded on one of the following instruments: a Waters ZMD single quad with electrospray (S/N mc350); a Perkin-Elmer SciX API 150ex spectrometer; a VG Quattro II triple quadrupole; a VG Platform II single quadrupole; or a Micromass Platform LCZ single quadrupole mass spectrometer (the latter three instruments were equipped with a pneumatically assisted electrospray interface (LC-MS)). ¹H NMR and ¹³C NMR measurements were performed on Varian 300, 400 and 500 spectrometers, operating at ¹H frequencies of 300, 400 and 500 MHz respectively, and at ¹³C frequencies of 75.5, 100.6 and 125.7 MHz respectively.

Rotamers may or may not be denoted in spectra depending upon ease of interpretation of spectra. Unless otherwise stated, chemical shifts are given in ppm with the solvent as internal standard.

Preparation A

3-(4-Cyanoanilino)propyl Benzenesulfonate

(i) 4-[(3-Hydroxypropyl)amino]benzonitrile

To 4-fluorobenzonitrile (30.29 g, 247.7 mmol, 1.0 eq), was added 3-amino-1-propanol (150 mL, 148.8 g, 1981.5 mmol, 8.0 eq). The mixture was stirred under nitrogen at room temperature (27° C.) until all of the solid had dissolved. The solution was heated (oil bath) to 77° C. and kept at this temperature for 7 hours, before being stirred at ambient temperature overnight (14 hours). Water (365 mL) was added, and the resultant cloudy solution was extracted with dichloromethane (365 mL, then 245 mL). The combined organic layers were washed with water (365 mL). The DCM solution of the product was dried by distillation: solvent (200 mL) was removed and replaced with fresh DCM (200 mL). More solvent (250 mL) was removed to bring the total solvent volume to 365 mL.

In a procedure slightly modified from that described above, the mixture of 4-fluorobenzonitrile and 3-amino-1-propanol can alternatively be heated to 80° C. for 5 hours. under nitrogen (instead of being stirred at ambient temperature, 77° C. and then ambient temperature again), after which it can be allowed to cool and have water added to it.

(ii) 3-(4-Cyanoanilino)propyl Benzenesulfonate

To the solution of 4-[(3-hydroxypropyl)amino]benzonitrile from step (i) above (assumed 43.65 g, 247.7 mmol, 1.0 eq) in dichloromethane (360 mL total solution volume) was added, sequentially, triethylamine (52 mL, 37.60 g, 371.55 mmol, 1.5 eq) and trimethylamine hydrochloride (11.89 g, 123.85 mmol, 0.5 eq) in one portion. The yellow solution was cooled to −20° C. (using a cold plate), and treated with a solution of benzenesulfonyl chloride (32 mL, 43.74 g, 247.7 mmol, 1.0 eq) in dichloromethane (220 mL, 5 vols with respect to the cyanoalcohol) via a pressure equalising dropping funnel. The solution was added portionwise such that the internal temperature did not exceed −14° C. The addition took 25 minutes to complete. The mixture was then stirred for 35 minutes at between −15 and −10° C.

Water (365 mL) was added and the temperature rose to 10° C. The mixture was cooled back to 0° C. and stirred vigorously for 15 minutes. The organic layer (volume 570 mL) was collected and distilled at atmospheric pressure to remove DCM (450 mL, pot temperature 40-42° C., still-head temperature 38-39° C.). Ethanol (250 mL) was added, and the solution was allowed to cool to below 30° C. before turning on the vacuum. More solvent was removed (40 mL was collected, pressure 5.2 kPa (52 mbar), pot and still-head temperatures were 21-23° C.), and the product gradually came out of solution. The distillation was stopped at this point, and more ethanol (50 mL) was added. The mixture was warmed (hot water bath at 50° C.) to 40° C. to dissolve all the solid, and water (90 mL) was added slowly via a dropping funnel. The solution was stirred slowly at room temperature (20° C.) overnight (15 hours), by which time some product had crystallised out. The mixture was cooled to −5° C. (ice/methanol bath) and stirred at this temperature for 20 minutes before collecting the pale yellow solid by filtration. The solid was washed with an ethano/water mixture (42 mL EtOH, 8 mL H₂O), and suction dried for 30 minutes before drying to constant weight in the vacuum oven (40° C., 72 hours). The mass of crude product obtained was 47.42 g (149.9 mmole, 60%).

Ethanol (160 mL, 8 vols) was added to the crude product (20.00 g, 63.22 mmol, 1.0 eq). The mixture was stirred under nitrogen and warmed to 40° C. using a hot water bath. On reaching this temperature, all of the solid had dissolved to give a clear, yellow solution. Water (60 mL, 3 vols) was added dropwise over a period of 10 minutes, whilst the internal temperature was maintained in the range 38-41° C. The water bath was removed, and the solution was allowed to cool to 25° C. over 40 minutes, by which time crystallisation had begun. The mixture was cooled to −5° C. over 10 minutes, then held at this temperature for a further 10 minutes. The pale yellow solid was collected by filtration, suction dried for 10 minutes, then dried to constant weight in a vacuum oven (40° C., 15 hours). The mass of title compound obtained was 18.51 g (58.51 mmol, 93% (from the crude product)).

Preparation B

3,3-Dimethyl-1-(9-oxa-3,7-diazabicyclo[3.3.1]non-3-yl)-2-butanone

(i) N,N-Bis(2-oxiranylmethyl)benzenesulfonamide

Water (2.5 L, 10 vol.) followed by epichlorohydrin (500 mL, 4 eq.) were added to benzenesulfonamide (250 g, 1 eq.). The reactants were heated to 40° C. Aqueous sodium hydroxide (130 g in 275 mL of water) was added such that the temperature of the reaction remained between 40° C. and 43° C. This took approximately 2 hours. (The rate of sodium hydroxide addition needs to be slower at the start of the addition than at the end in order to keep within the temperature range stated.) After the addition of sodium hydroxide was complete, the reaction was stirred at 40° C. for 2 hours, then at ambient temperature overnight. The excess epichlorohydrin was removed as a water azeotrope by vacuum distillation (ca. 4 kPa (40 mbar), internal temp 30° C.), until no more epichlorohydrin distilled. Dichloromethane (1L) was added and the mixture stirred rapidly for 15 minutes. The phases were allowed to separate (this took 10 minutes although totally clear phases are obtained after standing overnight). The phases were separated and the dichloromethane solution used in the subsequent step below.

¹H NMR (400 MHz, CDCl₃): δ 2.55-2.65 (2H, m), 2.79 (2H, t, J 4.4), 3.10-3.22 (4H, m), 3.58-3.73 (2H, m), 7.50-7.56 (2H, m), 7.58-7.63 (1H, m), 7.83-7.87 (2H, m).

(ii) 5-Benzyl-3,7-dihydroxy-1-phenylsulfonyl-1,5-diazacyclooctane

IMS (2.5 L, 10 vol) was added to the dichloromethane solution from step (i) above. The solution was distilled until the internal temperature reached 70° C. Approximately 1250 mL of solvent was collected. More IMS (2.5 L, 10 vol) was added followed by benzylamine (120 mL, 0.7 eq.) in one portion (no exotherm seen), and the reaction was heated at reflux for 6 hours (no change from 2 hour sampling point). More benzylamine was added (15 mL) and the solution was heated for a further 2 hours. The IMS was distilled off (ca. 3.25 L) and toluene was added (2.5 L). More solvent was distilled (ca. 2.4 L) and then further toluene added (1 L). The head temperature was now 110° C. A further 250 mL of solvent was collected at 110° C. Theoretically, this left the product in ca. 2.4 L of toluene at 110° C. This solution was used in the next step.

¹H NMR (400 MHz, CDCl₃): δ 7.83-7.80 (4H, m, ArH), 7.63-7.51 (6H, m, ArH), 7.30-7.21 (10H, ArH), 3.89-3.80 (4H, m, CH(a)+CH(b)), 3.73 (2H, s, CH₂Ph(a)), 3.70 (2H, s, CH₂Ph(b)), 3.59 (2H, dd, CHHNSO₂Ar(a)), 3.54 (2H, dd, CHHNSO₂Ar(b)), 3.40 (2H, dd, CHHNSO₂Ar(b)), 3.23 (2H, dd, CHHNSO₂Ar(a)), 3.09-2.97 (4H, m, CHHNBn(a)+CHHNBn(b)), 2.83 (2H, dd, CHHNBn(b)), 2.71 (2H, dd, CHHNBn(a)) (Data taken from purified material comprising a 1:1 mixture of trans- (a), and cis-diol (b))

(iii) 3-Benzyl-7-(phenylsulfonyl)-9-oxa-3,7-diazabicyclo[3.3.1]nonane

The toluene solution from the previous step (ii) above was cooled to 50° C. Anhydrous methanesulfonic acid (0.2 L) was added. This caused a temperature rise from 50° C. to 64° C. After 10 minutes, methanesulfonic acid was added (1 L) and the reaction heated to 1 10° C. for 5 hours. Toluene was then distilled from the reaction; 1.23 L was collected. (Note that the internal temperature should not be allowed higher than 110° C. at any stage otherwise the yield will be decreased.) The reaction was then cooled to 50° C. and a vacuum applied to remove the rest of the toluene. Heating to 110° C. and 65 kPa (650 mbar) allowed a further 0.53 L to be removed. (If the toluene can be removed at a lower temperature and pressure then that is beneficial.) The reaction was then left to cool to 30° C. and deionised water (250 mL) was added. This caused the temperature to rise from 30° C. to 45° C. More water (2.15 L) was added over a total time of 30 minutes such that the temperature was less than 54° C. The solution was cooled to 30° C. and then dichloromethane (2 L) was added. With external cooling and rapid stirring, the reaction mixture was basified by adding aqueous sodium hydroxide (10 M, 2 L) at a rate that kept the internal temperature below 38° C. This took 80 minutes. The stirring was stopped and the phases separated in 3 minutes. The layers were partitioned. IMS (2 L) was added to the dichloromethane solution and distillation started. Solvent (2.44 L) was collected until the head temperature reached 70° C. Theoretically, this left the product in 1.56 L of IMS. The solution was then allowed to cool to ambient temperature overnight with slow stirring. The solid product that precipitated was filtered and washed with IMS (0.5 L) to give a fawn-coloured product that, on drying at 50° C., in vacuum, gave 50.8 g (8.9% over 3 steps). 20.0 g of this product was dissolved in acetonitrile (100 mL) at reflux to give a pale yellow solution. After cooling to ambient temperature, the crystals that formed were collected by filtration and washed with acetonitrile (100 mL). The product was dried in vacuo at 40° C. for 1 hour to give 17.5 g (87%) of sub-title compound.

¹H NMR (400 MHz, CDCl₃): δ 7.18-7.23 (10H, m), 3.86-3.84 (2H, m), 3.67 (2H, d), 3.46 (2H, s), 2.91 (2H, d), 2.85 (2H, dd), 2.56 (2H, dd)

(iv) 3-Benzyl-9-oxa-3,7-diazabicyclo[3.3.1]nonane×2 HCl

Concentrated hydrobromic acid (1.2 L, 3 rel. vol.) was added to solid 3-benzyl-7-(phenylsulfonyl)-9-oxa-3,7-diazabicyclo[3.3.1]nonane (400 g, see step (iii) above) and the mixture was heated to reflux under a nitrogen atmosphere. The solid dissolved in the acid at 95° C. After heating the reaction for 8 hours, HPLC analysis showed that the reaction was complete. The contents were cooled to room temperature. Toluene (1.2 L, 3 rel. vol.) was added and the mixture stirred vigorously for 15 minutes. Stirring was stopped and the phases were partitioned. The toluene phase was discarded along with a small amount of interfacial material. The acidic phase was returned to the original reaction vessel and sodium hydroxide (10 M, 1.4 L, 3.5 rel. vol.) was added in one portion. The internal temperature rose from 30° C. to 80° C. The pH was checked to ensure it was >14. Toluene (1.6 L, 4 rel. vol.) was added and the temperature fell from 80° C. to 60° C. After vigorous stirring for 30 minutes, the phases were partitioned. The aqueous layer was discarded along with a small amount of interfacial material. The toluene phase was returned to the original reaction vessel, and 2-propanol (4 L, 10 rel. vol.) was added. The temperature was adjusted to between 40° C. and 45° C. Concentrated hydrochloric acid (200 mL) was added over 45 minutes such that the temperature remained at between 40° C. and 45° C. A white precipitate formed. The mixture was stirred for 30 minutes and then cooled to 7° C. The product was collected by filtration, washed with 2-propanol (0.8 L, 2 rel vol.), dried by suction and then further dried in a vacuum oven at 40° C. Yield=297 g (91%).

¹H NMR (CD₃OD+4 drops D₂O): δ 2.70 (br d, 2H), 3.09 (d, 2H), 3.47 (br S, 4H), 3.60 (s, 2H), 4.12 (br s, 2H), 7.30-7.45 (m, 5H). API MS: m/z=219 [C₁₃H₁₈N₂O+H]⁺.

(v) 3,3-Dimethyl-1-[9-oxa-7-(phenylmethyl)-3,7-diazabicyclo[3.3.1]non-3-yl]-2-butanone

Water (500 mL, 5 vol.) followed by 1-chloropinacolone (45.8 mL, 1 eq.) were added to sodium bicarbonate (114.2 g, 4 eq.). A solution of 3-benzyl-9-oxa-3,7-diazabicyclo[3.3.1]nonane×2 HCl (100.0 g; see step (iv) above) in water (300 mL, 3 vol.) was added slowly, so that the evolution of carbon dioxide was controlled (20 mins.). The reaction mixture was heated at 65 to 70° C. for 4 hours. After cooling to ambient temperature, dichloromethane (400 mL, 4 vol.) was added and, after stirring for 15 minutes, the phases were separated. The aqueous phase was washed with dichloromethane (400 mL, 4 vol.) and the organic extracts combined. The solution was distilled and solvent collected (550 mL). Ethanol (I L) was added and the distillation continued. Further solvent was collected (600 mL). Ethanol (1 L) was added and the distillation continued. Further solvent was collected (500 mL) (the head temperature was now 77° C.). This solution (theoretically containing 1150 mL of ethanol) was used directly in the next step.

¹H NMR (400MHz, CDCl₃): δ 1.21 (9H, s), 2.01-2.59 (2H, m), 2.61-2.65 (2H, m), 2.87-2.98 (4H, m), 3.30 (2H, s), 3.52 (2H, s), 3.87 (2H, br s), 7.26 (2H, d,J7.6), 7.33 (1H, dd,J7.6, 7.6), 7.47 (2H, d,J7.6).

(vi) 3,3-Dimethyl-1-(9-oxa-3,7-diazabicyclo[3.3.1]non-3-yl)-2-butanone

Palladium on charcoal (44 g, 0.4 wt. eq. of 61% wet catalyst, Johnson Matthey Type 440L) was added to the ethanol solution from the previous step (v) above. The mixture was hydrogenated at 400 kPa (4 bar). The reaction was considered complete after 5 hours. The catalyst was removed by filtration and washed with ethanol (200 mL). The combined ethanol filtrates were used without further purification. Solution assay gave 61.8 g of title product in ethanol (theoretically 1.35 L; measured 1.65 L). A portion of the product was isolated and purified. Analysis was performed on the purified product.

¹H NMR (300 MHz, CDCl₃): δ 1.17 (9H, s), 2.69 (2H, dt, J 11.4, 2.4), 2.93 (2H, d, J 10.8), 3.02 (2H, d, J 13.8), 3.26 (2H, s), 3.32 (2H, dt, J 14.1), 3.61 (2H, br s).

This reaction may also be performed using a lower weight ratio of catalyst to benzylated starting material. This may be achieved in several different ways, for example by using different catalysts (such as Pd/C with a metal loading different from that in the Type 440L catalyst employed above, or Rh/C) and/or by improving the mass transfer properties of the reaction mixture (the skilled person will appreciate that improved mass transfer may be obtained, for example, by performing the hydrogenation on a scale larger than that described in the above reaction). Using such techniques, the weight ratio of catalyst to starting material may be reduced below 4:10 (e.g. between 4:10 and 1:20.).

### Example 1

4-({3-[7-(3,3-Dimethyl-2-oxobutyl)-9-oxa-3,7-diazabicyclo[3.3.1]non-3-yl]propyllamino)benzonitrile, Benzenesulfonic Acid Salt

To an ethanol solution (total volume 770 mL, approx. 20 vols with respect to the amine) of 3,3-dimethyl-1-(9-oxa-3,7-diazabicyclo[3.3.1]non-3-yl)-2-butanone (assumed 34.97 g (verified by assay), 154.5 mmol, 1.0 eq; see Preparation B above) was added 3-(4-cyanoanilino)propyl benzenesulfonate (49.05 g, 154.52 mmol, 1.0 eq; see Preparation A above) in one portion. The resultant mixture was heated at 74° C. for 6 hours, then stirred at room temperature (20° C.) for 65 hours (over the weekend; the skilled person will appreciate that the reaction will also succeed without this prolonged stirring at room temperature). Ethanol (370 mL) was removed, and water (200 mL) was added (this gave a 2:1 EtOH:H₂O mixture, total volume 600 mL). Upon adding the water, the pot temperature fell from 80° C. to 61° C. The solution was re-heated to 70° C., then allowed to cool naturally to ambient temperature overnight (19 hours), whilst stirring slowly. A solid was observed at this stage. The mixture was cooled to 0° C. and then stirred at this temperature for 15 minutes before collecting the off-white solid by filtration. The solid was washed with a cold 2:1 mixture of ethanol:water (150 mL), suction dried for 1.25 hours, then oven-dried (40° C., 20 hours). The mass of crude product obtained was 57.91 g (103.3 mmol, 60%).

The crude product was found to be 98.47% pure (as determined by HPLC analysis), and was recrystallised (using the procedure detailed below) to give the title compound in a purity of 99.75% (84% recovery).

Recrystallisation Procedure:

Ethanol (562 mL) and water (281 mL) were added to the crude product obtained above (56.2 g). The solution was heated to 75° C. All material dissolved at 55° C. The solution was held at 75° C. for 5 minutes, before being cooled to 5° C. over 1.5 hours. Precipitation started at 35° C. The cold solution was filtered and the collected precipitate was washed with ethanol: water (2:1, 168 mL). The solid material was sucked dry on the filter, before being dried in vacuo at 40° C. to give product (47.1 g, 84%).

Abbreviations

API=atmospheric pressure ionisation (in relation to MS)

br=broad (in relation to NMR)

d=doublet (in relation to NMR)

DCM=dichloromethane

DMF=N,N-dimethylfornamide

dd=doublet of doublets (in relation to NMR)

Et=ethyl

eq.=equivalents

h=hour(s)

HCl=hydrochloric acid

HPLC=high performance liquid chromatography

IMS=industrial methylated spirit

m=multiplet (in relation to NMR)

Me=methyl

min.=minute(s)

m.p.=melting point

MS=mass spectroscopy

Pd/C=palladium on carbon

q=quartet (in relation to NMR)

rt=room temperature

s=singlet (in relation to NMR)

t=triplet (in relation to NMR)

Prefixes n-, s-, i-, t- and tert- have their usual meanings: normal, secondary, iso, and tertiary.

1. A process for the preparation of a benzenesulfonic acid salt of a compound of formula I, 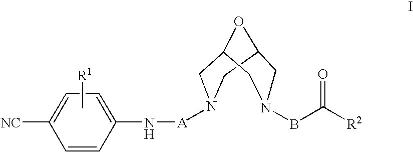
wherein R¹ represents H or cyano;
A represents (CH₂)₂₋₆;
B represents (CH₂)₁₋₄; and
R² represents C₁₋₆ alkyl, phenyl (which latter group is optionally substituted by one or two substituents selected from halo and methoxy) or benzodioxanyl;
which process comprises reaction of a compound of formula II, 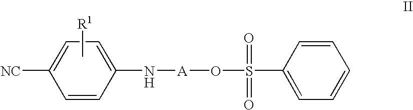
wherein R¹ and a are as defined above, with a compound of formula III, 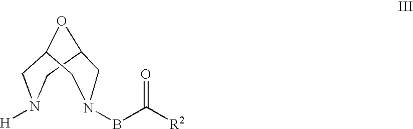
wherein B and R² are as defined above.

2. A process as claimed in claim 1, wherein, when R¹ represents cyano, it is located at the ortho-position relative to the group —N(H)-A-.

3. A process as claimed in claim 1, wherein R¹ represents H.

4. A process as claimed in any one of claims 1 to 3, wherein A represents (CH₂)₂₋₄.

5. A process as claimed in claim 4, wherein A represents n-propylene.

6. A process as claimed in any one of claims 1 to 5, wherein B represents (CH₂)₁₋₃.

7. A process as claimed in claim 6, wherein B represents CH₂.

8. A process as claimed in any one of claims 1 to 7, wherein R² represents benzodioxan-6-yl, 4-fluorophenyl, 4-bromophenyl, 4-methoxy-phenyl, 3,4-dimethoxyphenyl or C₁₋₄ alkyl.

9. A process as claimed in claim 8, wherein R² represents methyl or tert-butyl.

10. A process as claimed in claim 1, wherein R¹ represents H, A represents n-propylene, B represents CH₂ and R² represents tert-butyl.

11. A process as claimed in any one of claims 1 to 10, wherein the reaction is carried out in the presence of a solvent system.

12. A process as claimed in claim 11, wherein the solvent is ethanol.

13. A process as claimed in any one of claims 1 to 12, wherein the reaction is carried out at between 10 and 100° C.

14. A process as claimed claim 13, wherein the solvent is ethanol and the reaction is carried out at between 70 and 80° C.

15. A process as claimed in any one of claims 1 to 14, wherein the stoichiometric ratio of the compound of formula II to the compound of formula III is within the range of 3:2 to 2:3.

16. A process as claimed in claim 15, wherein the stoichiometric ratio is within the range 5:4 to 4:5.

17. A process as claimed in claim 16, wherein the stoichiometric ratio is 1:1.

18. A process as claimed in any one of claims 1 to 17, wherein the compound of formula I is subsequently precipitated from solution.

19. A process as claimed in claim 18, wherein the precipitation is facilitated by the addition of water to the reaction mixture.
